(19) United States
(12) Patent Application Publication
(10) Pub. No.: US 2017/0203053 A1
(43) Pub. Date: Jul. 20, 2017

(54) Visual-Assisted Insertion Device

(71) Applicant: Joseph Choate Burkett, Addison, TX (US)

(72) Inventor: Joseph Choate Burkett, Addison, TX (US)

(21) Appl. No.: 15/333,284

(22) Filed: Oct. 25, 2016

Related U.S. Application Data
(60) Provisional application No. 62/280,441, filed on Jan. 19, 2016.

(51) Int. Cl. A61M 5/42; A61M 25/02; A61B 6/12; A61B 5/055; A61B 8/08

(52) U.S. Cl. CPC A61M 5/427; A61B 5/055; A61B 8/0841; A61B 6/12; A61M 25/02; A61M 2025/0266

(57) ABSTRACT

A visual-assisted insertion device for ensuring accurate and repeatable needle insertion into the human body regardless of skill or training. A user of the visual-assisted insertion device may accurately perform inter-organ needle insertion by determining a desired needle-insertion point on an ultrasound display screen, wherein the insertion point and angle of the needle is automatically calculated and mechanically adjusted to correspond to the desired needle-insertion point. Accordingly, unsuccessful inter-organ needle insertion is minimized through a handheld, visual-assisted insertion device which simplifies the inter-organ needle insertion process.

### CROSS-REFERENCE TO RELATED APPLICATIONS

This application claims the benefit of U.S. Provisional Patent Application No. 62/280,441, filed Jan. 19, 2016, by the present inventor.

### TECHNICAL FIELD

The present apparatus relates generally to medicine; and, in particular, to needle-guided medical devices.

### BACKGROUND

In the medical field, the need often arises to inject or extract fluids from the body of a patient. Various medical procedures—including, without limitation, phlebotomies, intravenous medication administration, needle biopsies, epidurals, blood transfusions, and depot injections—all require the use of a hypodermic needle to gain access to interior organs. As such, the hypodermic needle is one of the most commonly-used tools in the practice of medicine.

Although so many common medical procedures require the use of a hypodermic needle, the effectiveness of such procedures is primarily reliant upon the proper insertion of the needle. Identifying structural locations under the surface of the skin is often difficult; and, at times, impossible. Variations in the thickness of skin, subcutaneous fat, and numerous other physical variations make it difficult, and sometimes dangerous, to insert needles in specific locations. Due to such variations, inter-organ access is not impervious to failure, even when conducted by master nurses, skilled phlebotomists, or other doctors who commonly insert hypodermic needles.

Accurate access to interior organs, especially smaller, peripheral veins, is critical to the health and wellbeing of patients. However, smaller, peripheral veins and organs are typically the most difficult to access, especially for patients in the greatest need of intravenous access. For example, renal patients, trauma patients, critically-ill patients, and dehydrated patients require intravenous access; however, due to their health complications, the peripheral veins may be constricted, scarred, or otherwise damaged, thereby preventing access to the lumen. Moreover, in emergency situations in which intravenous access is prevented, central or intraosseous line infusions—procedures with a higher frequency of life-threatening complications—must be performed.

Currently, procedures requiring needle insertion are typically performed manually by a physician or nurse. However, the variations in skill and knowledge between and among physicians and nurses, coupled with the inability to view underneath the skin, create a high frequency of error in needle placement.

To help remedy the problem, ultrasound has been used to help guide needle insertion. While ultrasound guidance has increased the likelihood of successful needle placement, it is replete with its own limitations. For example, very few physicians and nurses are taught or independently learn how to insert a needle using ultrasound guidance, as only a handful of physician residency specialties—such as surgeons, anesthesiologists, and emergency physicians—encourage the training of ultrasound-guided needle placement. Even with such encouragement, due to the limited focus of such specialties, ultrasound guidance training within such specialties is typically limited to central venous access (i.e., large vessels in the neck, chest, and thighs). Since these procedures involve large vessels and large needles, very few, if any, of these specialized physicians are trained to use the same technique to access smaller, peripheral vessels and organs.

Even with proper training, inter-organ access with ultrasound guidance is quite difficult in practice. Ultrasound-guided needle placement requires the practitioner to use the non-dominant hand for the ultrasound probe and the dominant hand for the needle, all while looking at an ultrasound image on a screen set off away from the patient. The multiple, independent variables increase the likelihood of error, even for the most skilled and dexterous practitioner.

Accordingly, there exists a need for a medical apparatus for accurate needle insertion, which simplifies the guided, needle-insertion process and can be utilized by practitioners with minimal training and skill. To date, no such medical apparatus exists.

### SUMMARY OF THE INVENTION

In accordance with the invention, unsuccessful needle insertion due to complicated insertion procedures or a lack of skill or training can be minimized through the utilization of a powered, handheld, visual-assisted insertion device (“VAID”). Such a VAID can produce accurate and repeatable needle insertion with minimal skill or training due to the simplification of the needle-insertion process.

In a particular embodiment of the invention, accurate and repeatable inter-organ needle placement can be achieved through operation of the VAID. In accordance with a particular embodiment of the invention, the VAID may feature an ultrasound imaging and display system integral to the handheld VAID, ensuring accuracy while also allowing a user to maintain focus on the desired needle placement. Additionally, the VAID may feature a mechanically-adjusted, interchangeable hypodermic needle cartridge wherein the user may manipulate the movement and placement of the needle with external controls on the VAID. In the present embodiment, the interchangeable hypodermic needle cartridge may be inserted in the side of the VAID and interact with and be controlled by a motor permanently within the VAID to move the cartridge and needle to an appropriate position for needle insertion. Furthermore, and in accordance with a particular embodiment of the invention, the VAID may feature a display screen integral to the VAID, which allows a user to accurately locate and track the movement and resulting placement of the needle. If desired, the VAID may also feature means for securing or stabilizing the VAID during the insertion process. Accordingly, through the utilization of the VAID, a single user may perform accurate and repeatable needle placement without physically touching and manipulating the needle, resulting in a higher needle-placement success rate and fewer risks of infection or other potential medical complications.

Aside from the common materials used to create hypodermic needles and ultrasound imaging systems, the VAID can be made from any desired material. However, particular embodiments of the invention may advantageously utilize hard plastic to reduce the weight of the VAID to facilitate easy utilization by a single user.

### BRIEF DESCRIPTION OF THE DRAWINGS

FIG. 1 is an external, side view of a visual-assisted insertion device embodying the principles of the present invention;

FIG. 2 is an internal, side view of a visual-assisted insertion device embodying the principles of the present invention;

FIG. 3 is a component view of an ultrasound display screen on a visual-assisted insertion device embodying the principles of the present invention;

FIG. 4 is a top, internal, component view of an interchangeable and disposable cartridge and corresponding motorized needle guide embodying the principles of the present invention;

FIG. 5 is a top, internal, component view of an interchangeable and disposable cartridge embodying the principles of the present invention;

FIG. 6 is an external rear view of a visual-assisted insertion device embodying the principles of the present invention;

FIG. 7 is an external perspective view of a visual-assisted insertion device embodying the principles of the present invention.

### DETAILED DESCRIPTION OF THE DRAWINGS

As noted above, inter-organ needle insertion is an oft-complicated and fallible process which requires specialized training to perform. Although ultrasound guidance systems have been used in attempts to make the process more accurate and repeatable, such systems require specialized training and ample skill, severely limiting the utility of ultrasound guidance systems. Currently, no known apparatus exists to remedy the complicated and fallible inter-organ needle insertion process.

The present invention is a visual-assisted insertion device (“VAID”) which produces accurate and repeatable needle insertion with minimal skill or training due to the simplification of the needle-insertion process and the utilization of imaging mechanisms for needle guidance. In the present embodiment, the VAID is powered on and the ultrasound display screen is illuminated. Then, the VAID is placed on the body at the targeted area and the ultrasound integral to the VAID locates venous structures. The ultrasound display screen shows a live view of venous structures beneath the skin and features a target and vertical guideline to guide placement. Once venous structures are identified in the primary view, a peripheral sweep bar is pressed up or down to sweep through all ultrasound views to ensure there are no overlaying, arteries and determine the horizontal angle of the blood vessel. By utilizing the ultrasound display screen, the user then aligns and centers the target on the desired location within the desired blood vessel by manipulating directional controls on the posterior controller. Once the desired target for needle placement is reached, internal components within the VAID automatically adjust the insertion angle based on the desired target point. The user then may press a needle-insertion button, which accurately inserts the needle at the desired target and angle. If the needle insertion is unsatisfactory, the user may stop the procedure by pressing a button to abort the procedure, which withdraws the needle and corresponding catheter to the rear-locked position and releases a catheter-securing mechanism from the skin. However, if the needle insertion is satisfactory, the user may press a trigger button which advances the catheter over the needle and locks the neck of the catheter into place through a catheter-secure mechanism. Once the catheter is locked into place, the needle is withdrawn and the VAID is released from the skin.

Additionally, in relation to the preceding example, the user may also advance the needle within the lumen of the blood vessel. To advance the needle within the lumen, the user changes the view on the ultrasound display screen to display an advanced section of the blood vessel. Within this view, the tip of the needle will no longer be visible and the target will be positioned at a different location. Similar to the initial insertion of the needle, the user may change the desired target for needle advancement while the VAID automatically calculates the angle and distance needed to reach the desired target. Once the desired target is selected, the user may again press the needle-insertion button to advance the needle to the desired location. This step may be repeated as many times as necessary.

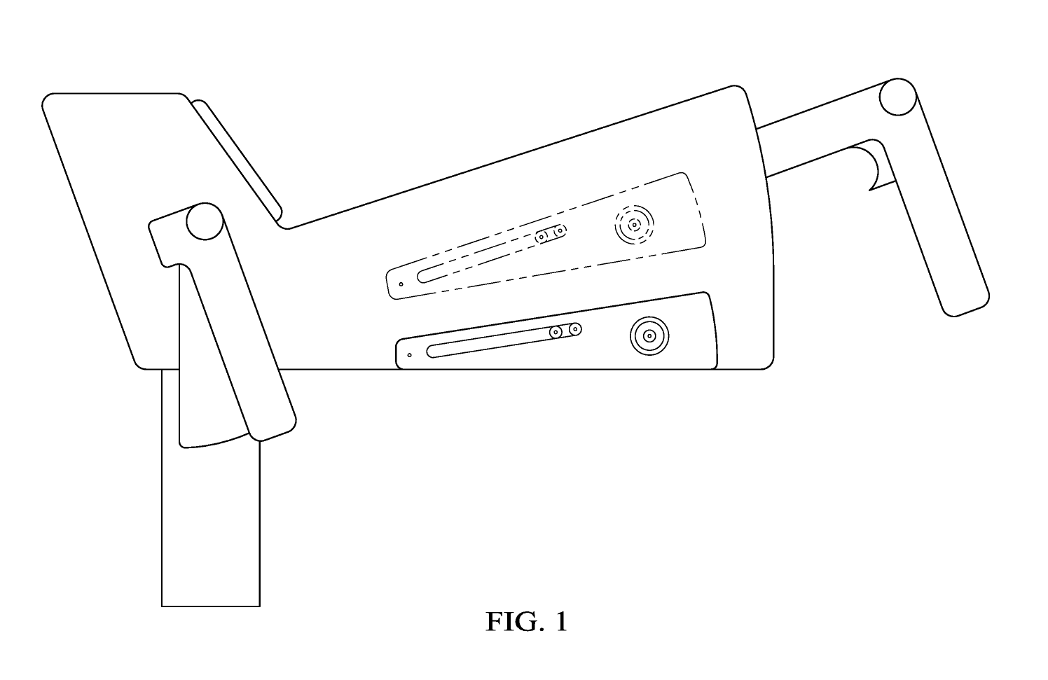

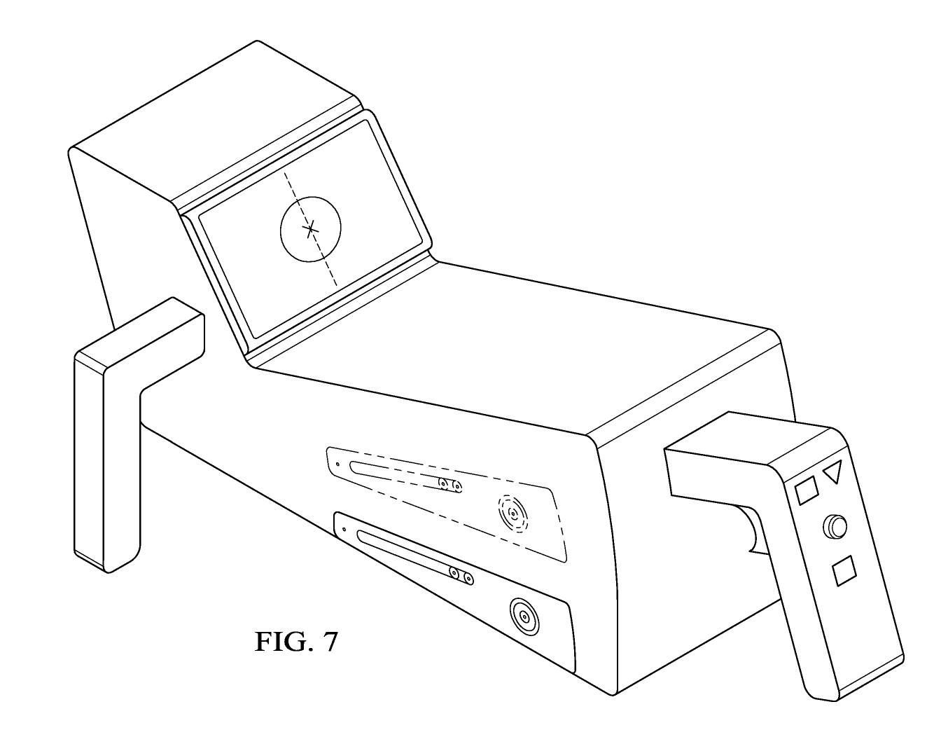

FIG. 1 and FIG. 7 generally depict a handheld VAID embodying the principles of the present invention. Particularly, FIG. 1 depicts an external, side view of a VAID embodying the principles of the present invention, whereas FIG. 7 depicts an external perspective view of a VAID embodying the principles of the present invention. In a particular embodiment, the exterior of the VAID may be comprised of a controller on the posterior of the VAID, a controller on the side of the VAID, and an ultrasound display screen or other imaging mechanism. In accordance with this particular embodiment, the posterior controller may feature a trigger button, a needle-insertion button, a directional button, and an abort button. Additionally, and in accordance with this particular embodiment, the ultrasound display screen may feature a target indicator and a vertical guideline, wherein the view on the ultrasound display screen is controlled by the side controller, which features a peripheral sweep bar and view-plane selector.

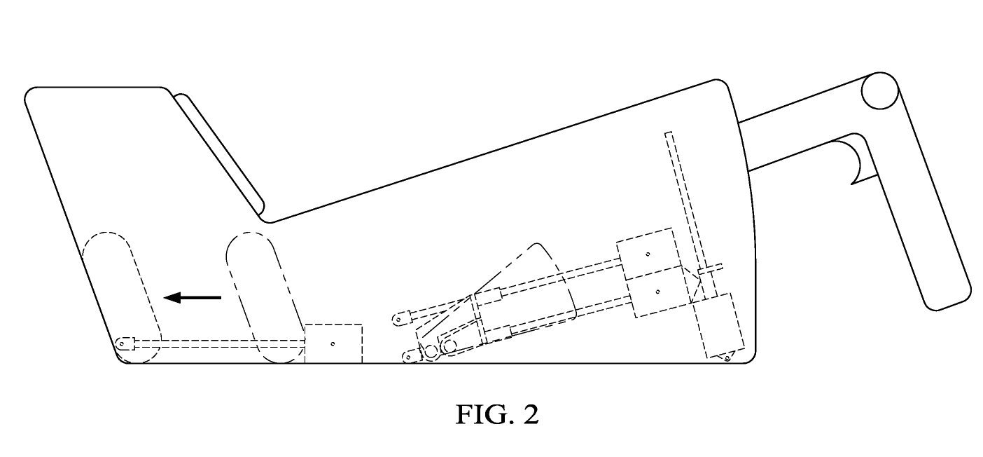

FIG. 2 depicts an interior view of a handheld VAID embodying the principles of the present invention. In a particular embodiment, the interior of the VAID may be comprised of a securing mechanism, an interchangeable hypodermic needle cartridge, a motor, a sensor, and an internal computer. In accordance with a principle of the present invention, the sensor may detect the angle of the hypodermic needle and send data to the internal computer. In such an embodiment, the internal computer may read the angular data from the sensor, calculate the necessary distance to move the needle to a desired target, and operate the motor to move the needle to the desired target. In a particular embodiment of the present invention, the hypodermic needle cartridge may feature an additional component, such as a catheter for intravenous access. In such an embodiment, once the needle has reached a desired location, the motor may move a catheter over the needle, locking the catheter within the securing mechanism which adheres the catheter to the skin through an adhesive component on the base of the securing mechanism.

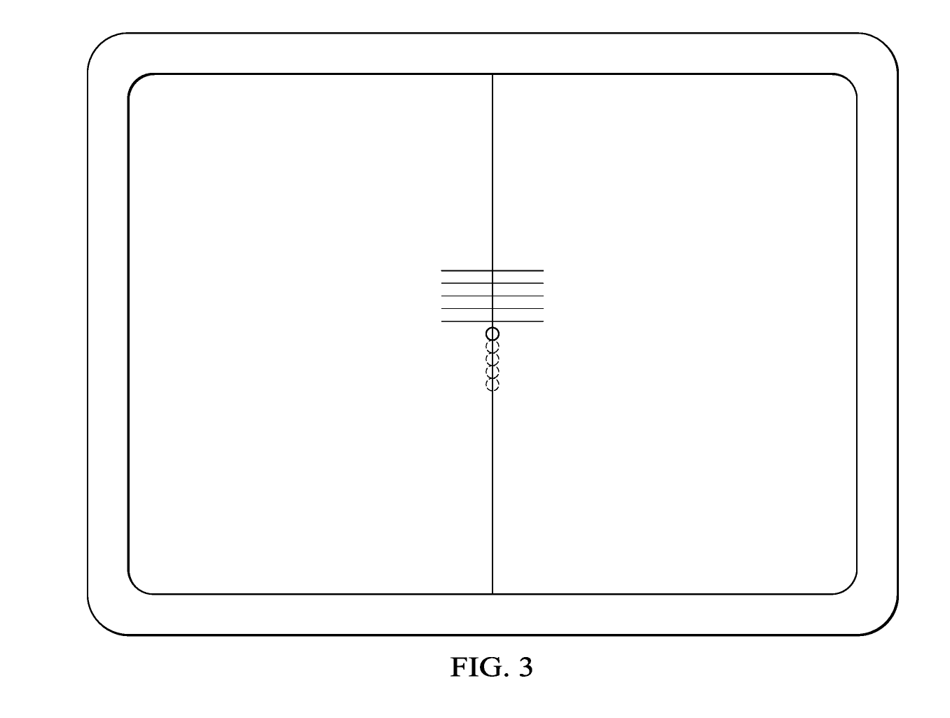

FIG. 3 depicts a component view of an ultrasound display screen on a VAID embodying the principles of the present invention. In a particular embodiment, the ultrasound display screen shows a live view of venous structures beneath the skin and features a target and vertical guideline to guide placement of a needle. In such an embodiment, the ultrasound display screen is controlled by a side controller which features a view-plane selector for changing the image on the ultrasound display and a peripheral sweep bar for sweeping through all ultrasound views to ensure there are no overlaying, arteries and determining the horizontal angle of a blood vessel.

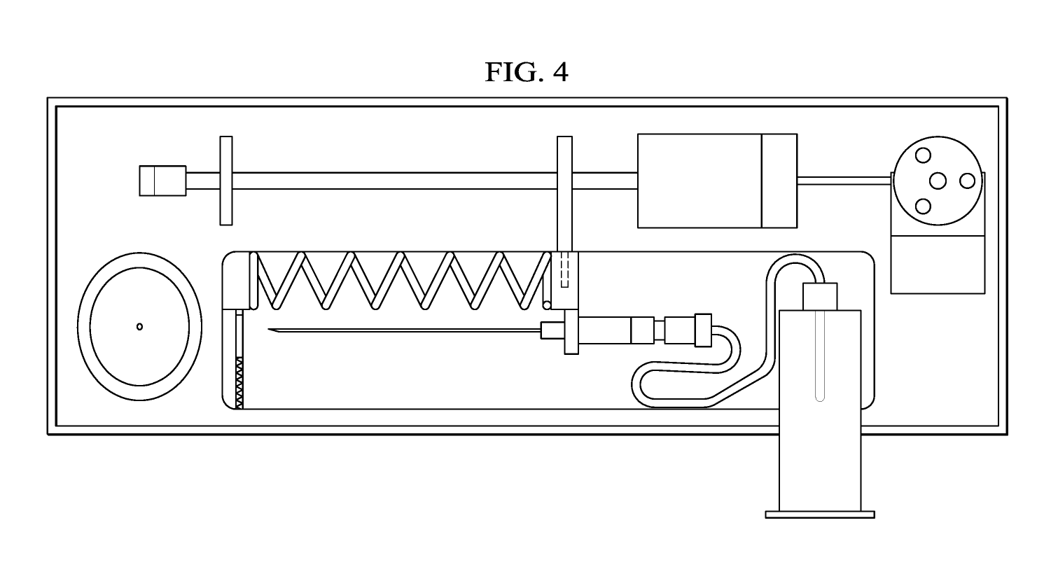

FIG. 4 depicts an interior component view of an interchangeable hypodermic needle cartridge interacting with a motorized needle guide in a VAID embodying the principles of the present invention. In a particular embodiment, the interchangeable hypodermic needle cartridge may insert into the side of the VAID and connect to a motorized needle guide integral within the VAID. The motorized needle guide may then raise and lower the interchangeable hypodermic needle cartridge to the appropriate angle and position for insertion of the hypodermic needle. In a particular embodiment, the interchangeable hypodermic needle cartridge may feature a detachable container for the drawing or insertion of fluids in the body. Once the hypodermic needle is in the desired position, the user may press a needle-insertion button to deploy the needle into the body.

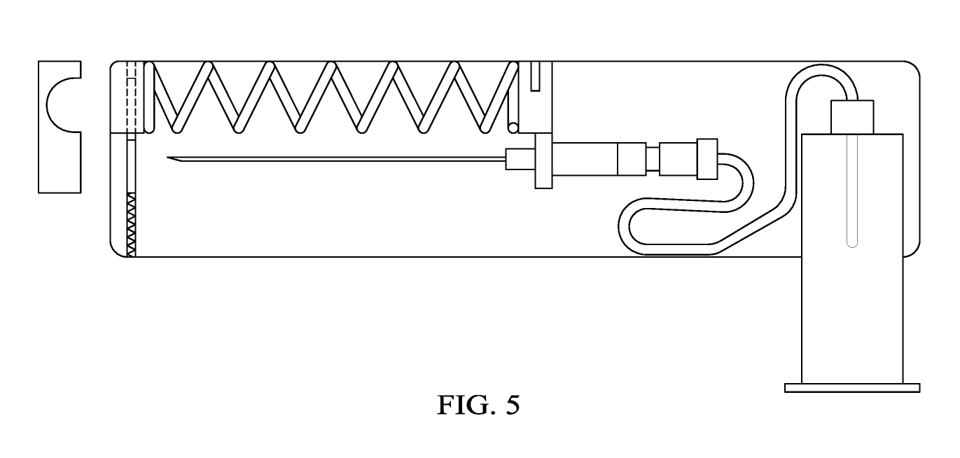

FIG. 5 depicts an interior component view of an interchangeable hypodermic needle cartridge embodying the principles of the present invention. In a particular embodiment, the interchangeable hypodermic needle cartridge may insert into the side of the VAID and connect to a motorized needle guide integral within the VAID. The interchangeable needle cartridge may also feature a detachable container for the drawing or insertion of fluids in the body.

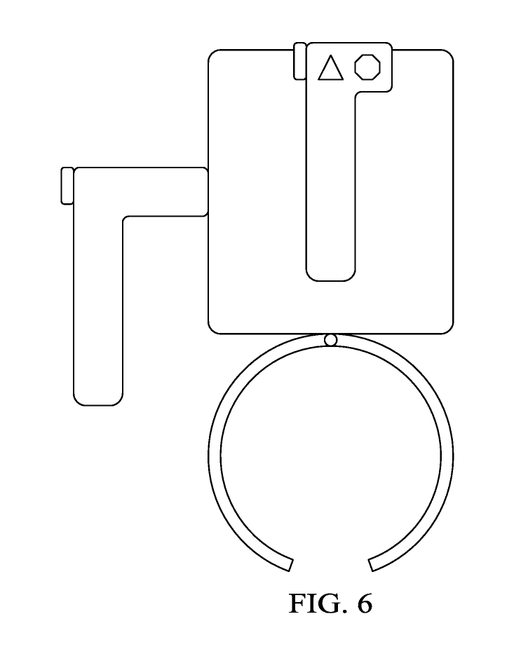

FIG. 6 depicts an external rear view of a VAID embodying the principles of the present invention. In a particular embodiment, the exterior of the VAID may be comprised of a controller on the posterior of the VAID and a controller on the side of the VAID. In accordance with this particular embodiment, the posterior controller may feature a trigger button, a needle-insertion button, a directional button, and an abort button. The side controller may feature a view-plane selector for changing the image on an ultrasound display and a peripheral sweep bar for sweeping through all ultrasound views to ensure there are no overlaying, arteries and determining the horizontal angle of a blood vessel. Accordingly, the posterior and side controllers may be utilized for the precise insertion, advancement, and removal of a needle within a lumen of a desired blood vessel.

The foregoing merely illustrates the principles of the present invention. Therefore, it will be appreciated that those skilled in the art will be able to devise numerous alternative arrangements that, while not shown or described herein, embody the principles of the invention and thus are within the spirit and scope of the invention.

What is claimed is:

1. A portable, needle-insertion apparatus comprising:
at least one imaging mechanism integral to said needle-insertion apparatus for providing an image of interior structures in a human body;
a display screen;
an interchangeable and disposable cartridge adapted to load into said needle-insertion apparatus;
one or more controllers integrally-connected to said needle-insertion apparatus;
a central processing unit; and
a stabilizing mechanism configured to stabilize and temporarily secure the needle-insertion apparatus to a human body during use of said needle-insertion apparatus.

2. The portable, needle-insertion apparatus of claim 1, wherein said interchangeable and disposable cartridge is further comprised of a hypodermic needle, catheter, and adhesive adapted to decouple from said interchangeable and disposable cartridge after insertion of said catheter and adhere to a surface to secure said catheter in place.

3. The portable, needle-insertion apparatus of claim 2, wherein said imaging mechanism is selected from the group consisting of: x-rays, ultrasound, gamma rays, and magnetic resonance imaging.

4. The portable, needle-insertion apparatus of claim 3, wherein said imaging mechanism is internally located within the apparatus.

5. The portable, needle-insertion apparatus of claim 4, wherein said imaging mechanism is positioned at an angle relative to the planar surface of a human body.

6. The portable, needle-insertion apparatus of claim 5, wherein said needle-insertion apparatus is further comprised of a housing with a distal end and a proximal end.

7. The portable, needle-insertion apparatus of claim 6, wherein one of said one or more controllers is attached to the proximal end of said housing of said needle-insertion apparatus.

8. The portable, needle-insertion apparatus of claim 7, wherein one of said one or more controllers is attached to a side of said housing proximate to the distal end of said housing of said needle-insertion apparatus.

9. The portable, needle-insertion apparatus of claim 8, wherein the controller attached to the proximal end of said housing is further comprised of a thumb wheel, trigger, and two or more pushbuttons.

10. The portable, needle-insertion apparatus of claim 9, wherein the controller attached to the proximal end of said housing is configured to adjust the insertion angle of said hypodermic needle, project and retract said hypodermic needle, and simultaneously retract said hypodermic needle and said catheter.

11. The portable, needle-insertion apparatus of claim 10, wherein the controller attached to a side of said housing is further comprised of a thumb wheel and squeeze grip.

12. The portable, needle-insertion apparatus of claim 11, wherein the controller attached to a side of said housing is configured to adjust the location of said imaging mechanism and stabilize said needle-insertion apparatus on a human body.

13. The portable, needle-insertion apparatus of claim 12, wherein said stabilizing mechanism is selected from the group consisting of: clamp, adhesive, sleeve, belt, and strap.

14. The portable, needle-insertion apparatus of claim 2, wherein said interchangeable and disposable cartridge is further comprised of a removable hub.
